(19) United States
(12) Patent Application Publication
(10) Pub. No.: US 2025/0086360 A1
(43) Pub. Date: Mar. 13, 2025

(54) SYSTEMS AND METHODS FOR TRAINING A MACHINE LEARNING MODEL FOR PREDICTIVE PLANT BREEDING USING PHENOMIC SELECTION BASED ON DIVERSE DATA STREAMS TO PREDICT GRAIN COMPOSITION

(71) Applicant: Benson Hill, Inc., St. Louis, MP (US)

(72) Inventors: Saeed Ahmadian, St. Louis, MO (US); Robert Koester, St. Louis, MO (US); Charles Pignon, St. Louis, MO (US); Craig Rolling, St. Louis, MO (US); Paul Skroch, St. Louis, MO (US); Yalda Zare, St. Louis, MO (US)

(21) Appl. No.: 18/725,468

(22) PCT Filed: Dec. 29, 2022
(86) PCT No.: PCT/US2022/054267

§ 371 Date: Jun. 28, 2024

Related U.S. Application Data
(60) Provisional application No. 63/295,751, filed on Dec. 31, 2021.

(51) Int. Cl. G06F 30/27; G06N 20/00

(52) U.S. Cl. CPC G06F 30/27; G06N 20/00

(57) ABSTRACT

The present disclosure is directed to methods (and associated systems) for training a machine learning model for predictive plant breeding using phenomic selection based on diverse data streams to predict grain composition comprising: collecting, with a processor, training data, stored in a database, from the group consisting essentially of phenomic data; selecting, with the processor, a machine learning model based on the training data, the machine learning model selected from the group comprising supervised learning models, unsupervised learning models, and combinations thereof; training, with the processor, the machine learning model using the training data from the database; and inputting, via the processor, a new set of phenotypic data from a plurality of grain bearing plants into the trained machine learning model to generate a predictive breeding crosses list ranked on an aggregate probability that a progeny of the cross will exhibit one or more desired phenotypic characteristics.

### CROSS REFERENCE TO RELATED APPLICATIONS

This application claims the benefit and priority of U.S. Provisional Patent Application Ser. No. 63/295,751 filed Dec. 31, 2021 entitled “Systems And Methods For Training A Machine-Learning Model For Predictive Plant Breeding Using Phenomic Selection Based On Diverse Data Streams To Predict Grain Composition,” the disclosure of which is hereby incorporated by references in their entirety.

### BACKGROUND

Genomics has been used for decades to develop crops for our food system, but most agricultural companies have focused almost exclusively on increasing the yield of a few crops, resulting in commodity ingredients and a food system based on the quantity of calories available. While focus on quantity is important, that focus resulted in lower nutrient density and changed flavors. Minimal diversity in ingredient options also led food manufacturers to add costly water- and energy-intensive processing steps, and additives like sugar and salt to make up for attributes that were muted in crops over time.

However, consumers are now demanding food choices with simpler ingredients that benefit their health and the health of our planet. Food- and diet-related health issues, including obesity and diabetes, are some of the most widespread health issues today and continue to increase. More than 65% of American adults are either overweight or have obesity and, according to the Centers for Disease Control and Prevention, approximately 90% of Americans do not eat the recommended daily amount of fruits and vegetables. Americans spend more on diet-related illnesses than on food itself.

Moreover, the current food system has a substantial environmental impact on the planet. According to an April 2020 report entitled “Agriculture and climate change” prepared by Mckinsey & Company, twenty-seven percent of total greenhouse gas emissions (e.g., methane and nitrous oxide) are caused by agriculture, with cattle and dairy cows alone contributing eight gigatons of carbon dioxide equivalent (GtCO2e) emissions in 2019. (Accessed Dec. 22, 2021 at https://www.mckinsey.com/˜/media/mckinsey/industries/agriculture/our%20insights/reducing %20agriculture%20emissions%20through%20improved%20farming%20practices/agriculture-and-climate-change.pdf.)

At the same time, demand for plant-based solutions to feed the world and improve the environment is growing. Consumers are open to changing their eating habits to minimize further harm to the environment. Moreover, people are actively trying to incorporate more plant based foods into their diets, especially protein alternatives found in the meat and dairy grocery store sections. NielsenIQ Sep. 9, 2021 article entitled “Growing demand for plant-based proteins” (Accessed Dec. 22, 2021 at https://nielseniq.com/global/en/insights/analysis/2021/examining-shopper-trends-in-plant-based-proteins-accelerating-growth-across-mainstream-channels/).

The largest commercial source of plant protein today is the soybean plant. Other plant-based protein crops include chickpeas, edamame, lentils, peanuts, and peas.

### Soybeans: Generally

Soybeans are believed to have originated on the Asian Continent (Glycine soja) where it is believed they were also first domesticated in China (Glycine max). Abstract, Hymowitz and Newell, Taxonomy of the genus Glycine, domestication and uses of soybeans. Econ Bot 35, 272-288 (1981). Soybeans are a common field crop with the largest producing countries including the United States, Brazil, Argentina, China, India, Paraguay, and Canada. In the United States in 2020 soybeans were primarily produced in the Western Corn Belt (48.7%), Eastern Corn Belt (32.7%), and the Midsouth (11.9%) with Illinois and Iowa being the largest producing states. Naeve and Miller-Garvin, United States Soybean Quality 2020 Annual Report (Published by the University of Minnesota with the support of the United Soybean Board).

Soybean plants produce seed-bearing pods, each generally having 2-4 seeds. The seeds are harvested and processed either for future planting (i.e., to produce additional soybean plants) or processed into dozens of products (e.g., bean curd, feed for livestock, flour, meal, oil (cooking and industrial)). Soy flours includes flour concentrates and isolates, which are the primary protein products of soy.

Soybean seeds are usually planted in rows in soil. According to the 2012 Illinois Soybean Production Guide, soybeans require 55-60° F. soil temperature, an air temperature of at least 68° F., about 25 inches of water, sufficient nitrogen and five months from germination to harvest.

The radical (or root) is the first structure to emerge from a germinating soybean seed. The hypocotyl is the seedling structure that emerges from the soil surface. As the hypocotyl emerges it forms a crook as it pulls the cotyledons (i.e., the plant's first leaves) from the soil. Then, the cotyledons can unfold and begin the process of photosynthesis. Once the cotyledons have emerged from the soil surface the plant is said to be at the VE stage of vegetative development. The VC (cotyledon) development stage occurs once two unifoliate (or single blade) leaves emerge from opposite sides of the main stem and no longer touch the cotyledons. The V1 (vegetative) development stage occurs once the unifoliate leaves are fully expanded establishing the first node. V2 is defined as the stage wherein a second node (with a trifoliate leaf (i.e. three or four leaflets per leaf)) has formed above the unifoliate node. With the formation of each subsequent node “n” (n=3, 4 . . . ) with fully developed leaves the plant is referred to as being in the Vn development stage. Soybean farmers typically refer to the leaves and stems as the canopy.

The length of time for these vegetative and reproductive stages (discussed below) depends on the plant's maturity group (“MG” (i.e., the length of time from planting to physical maturity), the soil and air temperatures, and day length. Soybeans are short-day plants (i.e., the soybean plant is triggered to flower as the day length decreases below some critical value, which differs among MGs). See, e.g., Purcell, Salmeron and Ashlock, “Chapter 2: Soybean Growth and Development” Arkansas Soybean Production Handbook (University of Arkansas Division of Agricultural Research & Extension, 2014 Update). Soybeans planted in Arkansas tend to be MG3 through MG6. Id. In Illinois, where soybeans may be grown in regions traditionally understood to be in MG2 through MG5, the 2012 Illinois Soybean Production Guide notes that MG 5 to MG 8 soybeans tend to be determinate (i.e., they cease vegetative growth when the main stem terminates in a cluster of mature pods) and MG 0 to MG 4.9 tend to be indeterminate (i.e. they develop leaves and flowers simultaneously after flowering begins).

Each soybean plant can produce a lot of flowers. The flowers are small and hidden underneath the leaves of the plant. The number of flowers produced depends upon the number of nodes on the main stem and branches with flower-bearing nodes. Not all flowers produce pods. For those flowers that do produce pods whether the resulting pod produces a full complement of seeds requires ample nitrogen, sugar, other nutrients, and favorable environmental conditions.

When a soybean plant begins to flower, it is referred to as being in its reproductive (R) growth stage. Soybeans are a normally self-pollinating crop, in fact, they have a perfect flower structure for self-pollination. Still, bees have been known to be attracted to soybean flowers and cross-pollinated plants. Where cross-pollination is desired breeders need to intervene to prevent self-pollination: the pistil of a soybean plant can become mature and the anthers can begin to shed pollen before the soybean flowers even bloom, breeders seeking to cross-pollinate need to be proactive.

Soybean plants have eight reproductive stages: R1 (beginning flowering/bloom (i.e., at least one flower)), R2 (full flowering/bloom (i.e., an open flower at one of the two uppermost nodes)), R3 (beginning pod (i.e., a pod measuring 3/16 inch at one of the four uppermost nodes)), R4 (full pod (i.e., a pod measuring ¾ inch at one of the four uppermost nodes)), R5 (beginning seed (i.e., a seed measuring ⅛ inch long in the pod at one of the four uppermost nodes)), R6 (full seed (i.e., a pod containing a green seed that fills the pod at one of the four uppermost nodes)), R7 (beginning maturity (i.e., one normal pod has reached mature pod color)), and R8 (full maturity (i.e., at least 95% of pods have reach full mature color)).

As the days get shorter and the temperatures get cooler, the leaves on soybean plants begin to turn yellow, they subsequently turn brown, fall off, and expose the matured pods of soybeans. The soybeans are now ready to be harvested using combines. The header on the front of the combine cuts and collects the soybean plants. The combine separates the soybeans from their pods and stems, and collects them into some container.

After harvesting the soybeans are processed. The soybeans are cleaned, heat dried, crushed and then flaked. Thereafter, the flake is further processed. The primary method for further processing is referred to as the extraction or solvent process, as it uses organic solvents (e.g. hexane) to recover the soybean oil and protein from the flake. Aside from its substantial use of solvents, this process consumes significant amounts of energy.

### Soybeans: Seed Varieties, Breeding, and Genetic Modification

Today, there are literally thousands of varieties of soybeans. These soybeans are the result of hundreds of years of selective breeding. Selective breeding is the process of selectively propagating plants with more desired traits (often called “phenotypes”) and eliminating plants with less desired phenotypes. Breeding generations are often designed F1, F2, etc, (wherein the “F” stands for “filial”). It may further involve crossing two plants to produce one or more new varieties.

Plant botanists have understood since the days of Gregor Mendel, that plants may exhibit dominant or recessive phenotypes/traits (e.g., seed shape, flower color, seed coat tint, pod shape, unripe pod color, flower location, and plant height). Through his experiments on pea plants, Mendel further taught that the genotype of a particular phenotype is not necessarily correlated because that phenotype may result from homozygous dominant, heterozygous, or homozygous recessive alleles. Where the phenotype is dominant, it will be exhibited by either of the first two zygosities. Whereas a recessive phenotype can only be exhibited by the third, homozygous recessive example.

Homozygous genotypes breed true from generation to generation, while heterozygous genotypes do not. Thus, after finding a desirable phenotype, plant breeders work to develop homozygosity in the population, and then release the resulting pure line as a new variety. For example, hybrid varieties are the result of crossing two homozygous, but unrelated pure lines of a species. The resulting F1 of the cross are all heterozygous. However, by F2 50% of the plants are either homozygous (dominant or recessive) and by F3 heterozygosity is reduced to 25%. Once a desired trait is found in homozygous plants, commercial quantities are produced by replanting the resulting seeds over several generations.

However, it takes time for each generation of plants to grow from seeds to adult plants and time to cross plants once they've produced reproductive organs. These and other practical constraints of biology are natural obstacles for traditional breeding programs and slow the advancement of potential commercial products through the phases of a traditional commercial product pipeline. Moreover, while the foregoing basic principles of plant genetics are relatively straightforward, the issue of creating a commercially-desirable variety is complicated by the fact that breeders cannot isolate a particular phenotype from other traits that might be present elsewhere in the plant's genome. Even in Mendel's pea experiments where he worked with only seven traits, each having at least two phenotypes (e.g., seed color: green or yellow) existing with the six other traits each of which also having multiple phenotypes the number of potential combinations explodes given all the ways the phenotypes of each trait can combine with the phenotypes of all the other traits. In the case of the soybean (i.e., Glycine max), its genome has approximately 1,100M base-pairs packaged into twenty chromosome pairs. Arumuganathan K, Earle ED. Nuclear DNA content of some important plant species. Plant molecular biology reporter. 1991 August; 9 (3): 208-18. Thus, there are an infinite number of potential genetic combinations within the soybean genome. As should be readily apparent, due to the sheer size of a plant's genome, the number of traits/phenotypes, and other practical constraints of biology, traditional plant breeding requires significant time to establish new phenotypes in a population.

Among the more desirable characteristics that have been selectively bred in soybeans are increased yield and increased tolerance to various potential environmental stressors (e.g., insects, drought). Unfortunately, according to the United States Soybean Quality 2020 Annual Report (conducted by Naeve and Miller-Garvin of the University of Minnesota), while soybean yields have significantly increased in the United States over the last thirty years, the amount of protein contained in those soybeans has substantially declined over the same time period.

### Using Machine Learning to Improve Agricultural Ingredients

While protein output in soybeans has been decreasing, the demand for plant-based protein has been growing. So much so that the demand will likely not be fully met using current breeding, genetic engineering, agronomic, and processing technologies. The current commodity food system can take on the order of six to ten years to improve crops with quality attributes, assuming the agricultural industry can even find the genetic synergy to create the right germplasm and then figure out how to best enable the desired breeding.

Machine learning (and other forms of artificial intelligence) are already being used to improve certain outcomes in agriculture. One key to successful machine learning is identifying the right types of data to gather and then using that data to train the right type of model. Another key may include identifying the wrong, unnecessary, or cumbersome data the inclusion of which is either unhelpful in developing the model or unnecessarily slows down or other makes the training process unnecessarily expensive without sufficient improvement of the model.

### SUMMARY OF THE DISCLOSURE

Phenomic selection is an emerging methodology which uses phenotypic data to build a model to predict future plant performance. Current uses of this methodology have been limited in terms of the phenotypes measured and the traits predicted. The novel phenomic selection disclosed herein utilizes a combination of phenotypes that have appear to have never been used previously in phenomic selection models. Moreover, phenomic selection has not previously been used to predict grain composition.

The present disclosure is directed to systems and methods for training a machine learning model for predictive plant breeding using phenomic selection based on diverse data streams to predict grain composition. The method comprises collecting, with a processor, training data, stored in a database, from the group consisting essentially of phenomic data; selecting, with the processor, a machine learning model based on the training data, the machine learning model selected from the group comprising supervised learning models, unsupervised learning models, and combinations thereof; training, with the processor, the machine learning model using the training data from the database; and inputting, via the processor, a new set of phenotypic data from a plurality of grain bearing plants into the trained machine learning model to generate a predictive breeding crosses list ranked on an aggregate probability that a progeny of the cross will exhibit one or more desired phenotypic characteristics.

The method may select the phenomic data from the group comprising: seed count, seed size, seed weight, and NIR spectra reflectance data from seed/grain. The group of phenomic data may further include analytical measurements of seed composition, which may be comprised of plant height, plant architecture, pod count, leaf size, photosynthetic capacity, root density, and days at each developmental stage.

Within certain approaches to the method the collecting of training data may further comprise gathering spectral reflectance imaging from overall plants, the phenomic data is further selected from the group comprising NDVI, NDRE, and senescence rate.

The machine learning model may comprise a plurality of stacked ML (machine learning) models. If so, the method further comprises a processor mediating between the plurality of stacked ML models to produce the aggregated predictive breeding crosses list.

These and other aspects of the disclosure will be further explained below.

### DRAWINGS

The Detailed Description is described with reference to the accompanying figures. The use of the same reference numbers in different instances in the description and the figures may indicate similar or identical items.

FIG. 1 is a flow diagram of the method for training a machine-learning model for predictive plant breeding using phenomic selection based on diverse data streams to predict grain composition.

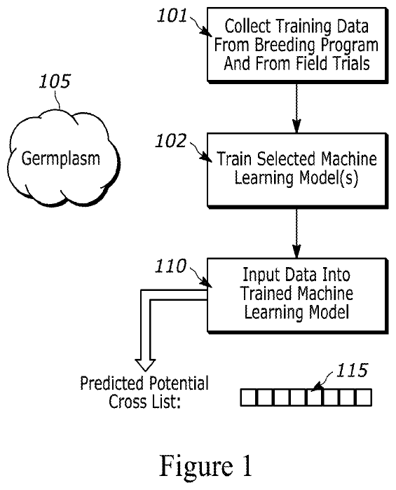

FIG. 2 is a diagram of features that may be used to train one example of the machine-learning model, which may include one or more types of machine learning models depending upon the type of feature data used.

FIG. 3 is a diagram illustrating the process of potential changes to one or more of the machine-learning models based on live data collection.

FIG. 4 is a block diagram illustration one potential system within which one or more of the inventive concepts disclosed in the present specification may be implemented.

### DETAILED DESCRIPTION

The present invention now will be described more fully hereinafter with reference to the accompanying drawings, which form a part hereof, and which show, by way of illustration, specific exemplary embodiments by which the invention may be practiced. This invention may, however, be embodied in many different forms and should not be construed as limited to the embodiments set forth herein; rather, these embodiments are provided so that this disclosure will be thorough and complete, and will fully convey the scope of the invention to those skilled in the art. Among other things, the present invention may be embodied as methods or devices. The following detailed description is, therefore, not to be taken in a limiting sense.

In the following detailed description of embodiments of the inventive concepts, numerous specific details are set forth in order to provide a more thorough understanding of the inventive concepts. However, it will be apparent to one of ordinary skill in the art that the inventive concepts within the disclosure may be practiced without these specific details. In other instances, certain well-known features may not be described in detail to avoid unnecessarily complicating the instant disclosure.

As used herein, the terms “comprises,” “comprising,” “includes,” “including,” “has,” “having,” or any other variation thereof, are intended to cover a non-exclusive inclusion. For example, a process, method, article, or apparatus that comprises a list of elements is not necessarily limited to only those elements but may include other elements not expressly listed or inherently present therein.

Unless expressly stated to the contrary, “or” refers to an inclusive or and not to an exclusive or. For example, a condition A or B is satisfied by anyone of the following: A is true (or present) and B is false (or not present), A is false (or not present) and B is true (or present), and both A and B are true (or present).

The term “and combinations thereof” as used herein refers to all permutations or combinations of the listed items preceding the term. For example, “A, B, C, and combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. A person of ordinary skill in the art will understand that typically there is no limit on the number of items or terms in any combination, unless otherwise apparent from the context.

In addition, use of the “a” or “an” are employed to describe elements and components of the embodiments herein. This is done merely for convenience and to give a general sense of the inventive concepts. This description should be read to include one or at least one and the singular also includes the plural unless it is obvious that it is meant otherwise.

The use of the terms “at least one” and “one or more” will be understood to include one as well as any quantity more than one, including, but not limited to, each of, 2, 3, 4, 5, 10, 15, 20, 30, 40, 50, 100, and all integers and fractions, if applicable, therebetween. The terms “at least one” and “one or more” may extend up to 100 or 1000 or more, depending on the term to which it is attached; in addition, the quantities of 100/1000 are not to be considered limiting, as higher limits may also produce satisfactory results.

Further, as used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

As used herein qualifiers such as “about,” “approximately,” and “substantially” are intended to signify that the item being qualified is not limited to the exact value specified, but includes some slight variations or deviations therefrom, caused by measuring error, manufacturing tolerances, stress exerted on various parts, wear and tear, and combinations thereof, for example.

As used herein, “components” may be analog or digital components that perform one or more functions. The term “component” may include hardware, such as a processor (e.g., microprocessor), a combination of hardware and software, and/or the like. Software may include one or more computer executable instructions that when executed by one or more components cause the component to perform a specified function. It should be understood that any and all algorithms described herein may be stored on one or more non-transitory memory. Exemplary non-transitory memory may include random access memory, read only memory, flash memory, and/or the like. Such non-transitory memory may be electrically based, optically based, and/or the like.

Methods disclosed herein include conferring desired traits to plants, for example, using plant breeding techniques and various crossing schemes, etc. These methods are not limited as to certain mechanisms of how the plant exhibits and/or expresses the desired trait. In certain nonlimiting embodiments, the desired trait is conferred to a plant by crossing two plants to create offspring that express the desired trait. It is expected that users of these teachings will employ a broad range of techniques and mechanisms known to bring about the expression of a desired trait in a plant. Thus, as used herein, conferring a desired trait to a plant is meant to include any process that causes a plant to exhibit a desired trait, regardless of the specific techniques employed.

As used herein, “fertilization” and/or “crossing” broadly includes bringing the genomes of gametes together to form zygotes but also broadly may include pollination, syngamy, fecundation and other processes related to sexual reproduction. Typically, a cross and/or fertilization occurs after pollen is transferred from one flower to another, but those of ordinary skill in the art will understand that plant breeders can leverage their understanding of fertilization and the overlapping steps of crossing, pollination, syngamy, and fecundation to circumvent certain steps of the plant life cycle and yet achieve equivalent outcomes, for example, a plant or cell of a soybean cultivar described herein. In certain embodiments, a user of this innovation can generate a plant of the claimed invention by removing a genome from its host gamete cell before syngamy and inserting it into the nucleus of another cell. While this variation avoids the unnecessary steps of pollination and syngamy and produces a cell that may not satisfy certain definitions of a zygote, the process falls within the definition of fertilization and/or crossing as used herein when performed in conjunction with these teachings. In certain embodiments, the gametes are not different cell types (i.e. egg vs. sperm), but rather the same type and techniques are used to effect the combination of their genomes into a regenerable cell. Other embodiments of fertilization and/or crossing include circumstances where the gametes originate from the same parent plant, i.e. a “self” or “self-fertilization”. While selfing a plant does not require the transfer pollen from one plant to another, those of skill in the art will recognize that it nevertheless serves as an example of a cross, just as it serves as a type of fertilization. Thus, methods and compositions taught herein are not limited to certain techniques or steps that must be performed to create a plant or an offspring plant of the claimed invention, but rather include broadly any method that is substantially the same and/or results in compositions of the claimed invention.

A “plant” refers to a whole plant, any part thereof, or a cell or tissue culture derived from a plant, comprising any of: whole plants, plant components or organs (e.g., leaves, stems, roots, etc.), plant tissues, seeds, plant cells, protoplasts and/or progeny of the same. A plant cell is a biological cell of a plant, taken from a plant or derived through culture of a cell taken from a plant.

A “population” means a set comprising any number, including one, of individuals, objects, or data from which samples are taken for evaluation, e.g. estimating QTL effects and/or disease tolerance. Most commonly, the terms relate to a breeding population of plants from which members are selected and crossed to produce progeny in a breeding program. A “population of plants” can include the progeny of a single breeding cross or a plurality of breeding crosses and can be either actual plants or plant derived material, or in silico representations of plants. The member of a population need not be identical to the population members selected for use in subsequent cycles of analyses nor does it need to be identical to those population members ultimately selected to obtain a final progeny of plants. Often, a “plant population” is derived from a single biparental cross but can also derive from two or more crosses between the same or different parents. Although a population of plants can comprise any number of individuals, those of skill in the art will recognize that plant breeders commonly use population sizes ranging from one or two hundred individuals to several thousand, and that the highest performing 5-20% of a population is what is commonly selected to be used in subsequent crosses in order to improve the performance of subsequent generations of the population in a plant breeding program.

“Crop performance” is used synonymously with “plant performance” and refers to of how well a plant grows under a set of environmental conditions and cultivation practices. Crop performance can be measured by any metric a user associates with a crop's productivity (e.g. yield), appearance and/or robustness (e.g. color, morphology, height, biomass, maturation rate), product quality (e.g. fiber lint percent, fiber quality, seed protein content, seed carbohydrate content, etc.), cost of goods sold (e.g. the cost of creating a seed, plant, or plant product in a commercial, research, or industrial setting) and/or a plant's tolerance to disease (e.g. a response associated with deliberate or spontaneous infection by a pathogen) and/or environmental stress (e.g. drought, flooding, low nitrogen or other soil nutrients, wind, hail, temperature, day length, etc.). Crop performance can also be measured by determining a crop's commercial value and/or by determining the likelihood that a particular inbred, hybrid, or variety will become a commercial product, and/or by determining the likelihood that the offspring of an inbred, hybrid, or variety will become a commercial product. Crop performance can be a quantity (e.g. the volume or weight of seed or other plant product measured in liters or grams) or some other metric assigned to some aspect of a plant that can be represented on a scale (e.g. assigning a 1-10 value to a plant based on its disease tolerance).

A “microbe” will be understood to be a microorganism, i.e. a microscopic organism, which can be single celled or multicellular. Microorganisms are very diverse and include all the bacteria, archaea, protozoa, fungi, and algae, especially cells of plant pathogens and/or plant symbionts. Certain animals are also considered microbes, e.g. rotifers. In various embodiments, a microbe can be any of several different microscopic stages of a plant or animal. Microbes also include viruses, viroids, and prions, especially those which are pathogens or symbionts to crop plants.

A “fungus” includes any cell or tissue derived from a fungus, for example whole fungus, fungus components, organs, spores, hyphae, mycelium, and/or progeny of the same. A fungus cell is a biological cell of a fungus, taken from a fungus or derived through culture of a cell taken from a fungus.

A “pest” is any organism that can affect the performance of a plant in an undesirable way. Common pests include microbes, animals (e.g. insects and other herbivores), and/or plants (e.g. weeds). Thus, a “pesticide” is any substance that reduces the survivability and/or reproduction of a pest, e.g. fungicides, bactericides, insecticides, herbicides, and other toxins.

“Tolerance” or improved tolerance in a plant to disease conditions (e.g. growing in the presence of a pest) will be understood to mean an indication that the plant is less affected by the presence of pests and/or disease conditions with respect to yield, survivability and/or other relevant agronomic measures, compared to a less tolerant, more “susceptible” plant. Tolerance is a relative term, indicating that a “tolerant” plant survives and/or performs better in the presence of pests and/or disease conditions compared to other (less tolerant) plants (e.g., a different soybean cultivar) grown in similar circumstances. As used in the art, tolerance is sometimes used interchangeably with “resistance”, although resistance is sometimes used to indicate that a plant appears maximally tolerant to, or unaffected by, the presence of disease conditions. Plant breeders of ordinary skill in the art will appreciate that plant tolerance levels vary widely, often representing a spectrum of more-tolerant or less-tolerant phenotypes, and are thus trained to determine the relative tolerance of different plants, plant lines or plant families and recognize the phenotypic gradations of tolerance.

A plant, or its environment, can be contacted with a wide variety of “agriculture treatment agents.” As used herein, an “agriculture treatment agent”, or “treatment agent”, or “agent” can refer to any exogenously provided compound that can be brought into contact with a plant tissue (e.g. a seed) or its environment that affects a plant's growth, development and/or performance, including agents that affect other organisms in the plant's environment when those effects subsequently alter a plant's performance, growth, and/or development (e.g. an insecticide that kills plant pathogens in the plant's environment, thereby improving the ability of the plant to tolerate the insect's presence). Agriculture treatment agents also include a broad range of chemicals and/or biological substances that are applied to seeds, in which case they are commonly referred to as “seed treatments” and/or seed dressings. Seed treatments are commonly applied as either a dry formulation or a wet slurry or liquid formulation prior to planting and, as used herein, generally include any agriculture treatment agent including growth regulators, micronutrients, nitrogen-fixing microbes, and/or inoculants. Agriculture treatment agents include pesticides (e.g. fungicides, insecticides, bactericides, etc.) hormones (abscisic acids, auxins, cytokinins, gibberellins, etc.) herbicides (e.g. glyphosate, atrazine, 2,4-D, dicamba, etc.), nutrients (e.g. a plant fertilizer), and/or a broad range of biological agents, for example a seed treatment inoculant comprising a microbe that improves crop performance, e.g. by promoting germination and/or root development. In certain embodiments, the agriculture treatment agent acts extracellularly within the plant tissue, such as interacting with receptors on the outer cell surface. In some embodiments, the agriculture treatment agent enters cells within the plant tissue. In certain embodiments, the agriculture treatment agent remains on the surface of the plant and/or the soil near the plant. In certain embodiments, the agriculture treatment agent is contained within a liquid. Such liquids include, but are not limited to, solutions, suspensions, emulsions, and colloidal dispersions. In some embodiments, liquids described herein will be of an aqueous nature. However, in various embodiments, such aqueous liquids that comprise water can also comprise water insoluble components, can comprise an insoluble component that is made soluble in water by addition of a surfactant, or can comprise any combination of soluble components and surfactants. In certain embodiments, the application of the agriculture treatment agent is controlled by encapsulating the agent within a coating, or capsule (e.g. microencapsulation). In certain embodiments, the agriculture treatment agent comprises a nanoparticle and/or the application of the agriculture treatment agent comprises the use of nanotechnology.

In certain embodiments, plants disclosed herein can be modified to exhibit at least one “desired trait”, and/or combinations thereof. The disclosed innovations are not limited to any set of traits that can be considered desirable, but nonlimiting examples include male sterility, herbicide tolerance, pest tolerance, disease tolerance, modified fatty acid metabolism, modified carbohydrate metabolism, modified seed yield, modified seed oil, modified seed protein, modified lodging resistance, modified shattering, modified iron-deficiency chlorosis, modified water use efficiency, and/or combinations thereof. Desired traits can also include traits that are deleterious to plant performance, for example, when a researcher desires that a plant exhibits such a trait in order to study its effects on plant performance.

The term “machine learning” generally refers to computer algorithms that may learn from pre-existing data and then make predictions about new data. Thus, machine-learning tools operate by building a model from example training data, which, for example, can be used to model an environment based on that training data and then make decisions or predictions without explicit instructions.

Different machine-learning tools may be used. Deep learning or deep structured learning is a type of machine learning that can use artificial neural networks (e.g., inspired by biological systems) with representation learning. Representation learning is a set of techniques that allows a system to automatically discover representations needed to detect features in future sets of data.

Data for predictor and target variables for phenomic selection models are collected. Predictor variable data are preferably collected at various growth stages (e.g., from before VE through R8) from indoor, early-stage plant breeding operations. The predictor variable data collected from indoor, early-stage plant breeding operations may include, but are not limited to,
(I) seed count (total number of seeds produced by a particular plant); seed size; seed weight; NIR spectra reflectance data from seed/grain (which may be used to estimate, for example, protein and oil concentrations and moisture content);
(II) analytical measurement of seed/grain composition (e.g., fiber content, water content, oleic acid content, oligosaccharides (e.g., raffinose and stachyose) content, saponins content, isoflavones content, PUFA content, Hexanal content, Hexanol content, and molecular markers);
(III) plant height; plant architecture (e.g., viny, bushy, etc.); pod count; leaf size; photosynthetic capacity; root density; and days at each developmental stage; and (IV) spectral reflectance imaging of the overall plant and selected plant surfaces (e.g., radical, cotyledons, leaves, stems, pods, flowers (e.g., pistil, stamen, anther)) in the visible (i.e., Red, Green, Blue) and invisible (e.g., ultra-violet, infrared, near-infrared) ranges toward calculating various measurements including normalized difference vegetation index (NDVI, calculated as (NIR-Red)/(NIR+Red)—which may provide an indication of overall plant stress, NDRE (Normalized Difference Red Edge, calculated as (NIR−RE)/(NIR+RE), where RE is measured at 715 nm (i.e., the boundary for light absorption by chlorophyll in the red visible region), and senescence rate.
   The data needed to calculate NDVI and/or NDRE may be gathered using an infrared camera/image sensor and a typical RGB camera/image sensor both of which are found in multispectral camera/image sensors.

Similar predictor variable data may also be collected at various growth stages in agricultural fields. Additional predictor variable data may also be collected (especially in agricultural fields) using diverse technologies. Those diverse technologies may include, but are not limited to, reflectance spectra from field plot hyperspectral images and 3D LIDAR point clouds. The hyperspectral reflectance data may include, but is not limited to data related to plant biomass, flower detection, and leaf area. The data may be processed to extract additional features from the raw data, such as NDVI from the hyperspectral images and plant height from the lidar point clouds.

The target variables are primarily protein concentration, but may also include lower priority traits such as oil content and grain yield. Grain composition is collected by measuring the NIR reflectance of grain samples from these plots, the measured values may be used to estimate grain composition traits such as protein and oil concentration, and moisture content. Yield values may be collected by combine-harvesting yield trial plots from later stages of the breeding pipeline.

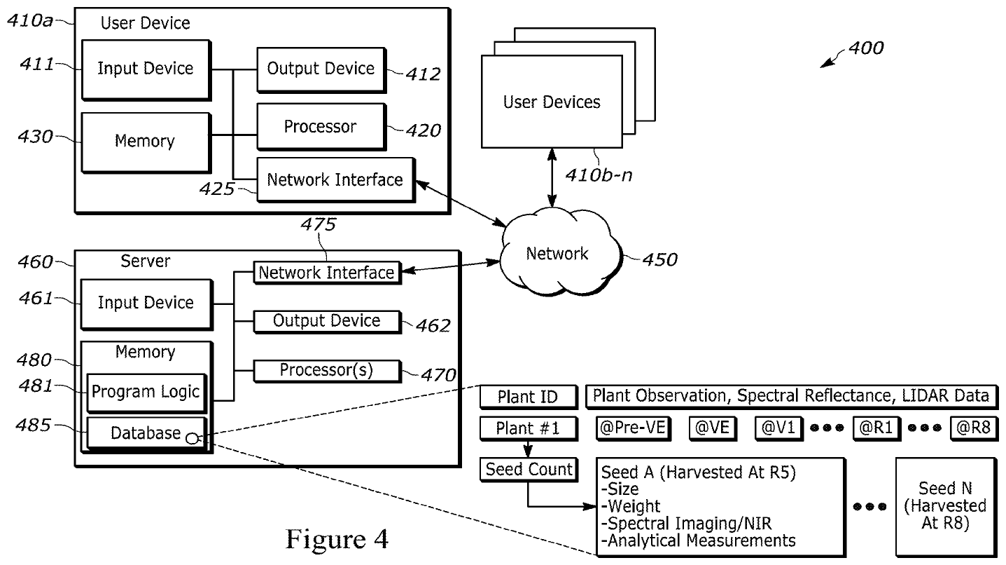

All of the data may be compiled into database 485 (see FIG. 4) and may undergo standard data processing, including, for instance, outlier removal, mean centering and standardization.

The ML model is built using standard machine learning design protocols including, but not limited model testing, feature selection and hyperparameter tuning. The models may be either supervised or unsupervised, although a hybrid of these approaches is also possible.

In “supervised learning,” a “teacher” presents the computer with the desired outputs given a set of example inputs. This is generally thought to involve classification and regression, which can be accomplished using one or more approaches including, but not limited to, decision trees, ensembles (e.g. Random Forest), nearest neighbors algorithm, linear regression, gBLUP (genomic best linear unbiased prediction), lasso (least absolute shrinkage and selection operator), lasso LARS, Ridge regression, Elastic Net, Naive Bayes, Artificial neural networks, logistic regression, perceptron, Relevance vector machine (RVM), and Support vector machine (SVM). Generally, the approach to supervised learning used depends on the data set, among other issues involved in this choice is the amount training data available, the dimensionality and heterogeneity of that data, redundancy in that data, the interrelations between data elements, and the amount of noise present in the output.

In “unsupervised learning,” the computer is left to find any naturally occurring patterns within the training data. This can be accomplished by using one or more approaches including, but not limited to, clustering (i.e., automatically grouping the training examples into categories with similar features), anomaly detection, principal component analysis (i.e., automatically identifying features that are most useful for discriminating between different training examples and then discarding the rest), self-organizing feature maps, and latent variable models. Clustering methods include hierarchical clustering, k-means, mixture models (i.e., a probabilistic model that represents the presence of subpopulations within an overall population), DBSCAN (density-based spatial clustering of applications with noise), expectation-maximization, BIRCH, and CURE.

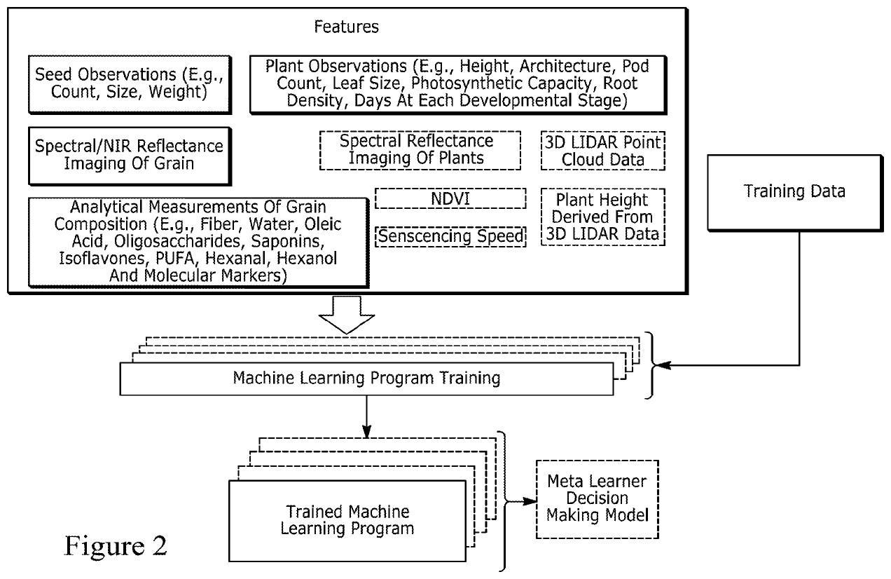

As illustrated in FIG. 2, one or more of the foregoing supervised and unsupervised machine learning approaches may be used by the present system and methods in parallel or seriatim using the same training data or subsets thereof. Where subsets are used the scope of any such subset may be selected for use with the particularly selected training data within that subset with reference to the pluses and minuses of one or more of the particular approaches to machine learning. Where multiple machine learning approaches are used in parallel (i.e., stacked) a meta learner ML decision making model is preferably introduced to mediate between the probability assessments provided by the multiple machine learning models toward providing a single list of recommended actions (e.g., desirable plant crosses, gene editing targets, crop management techniques).

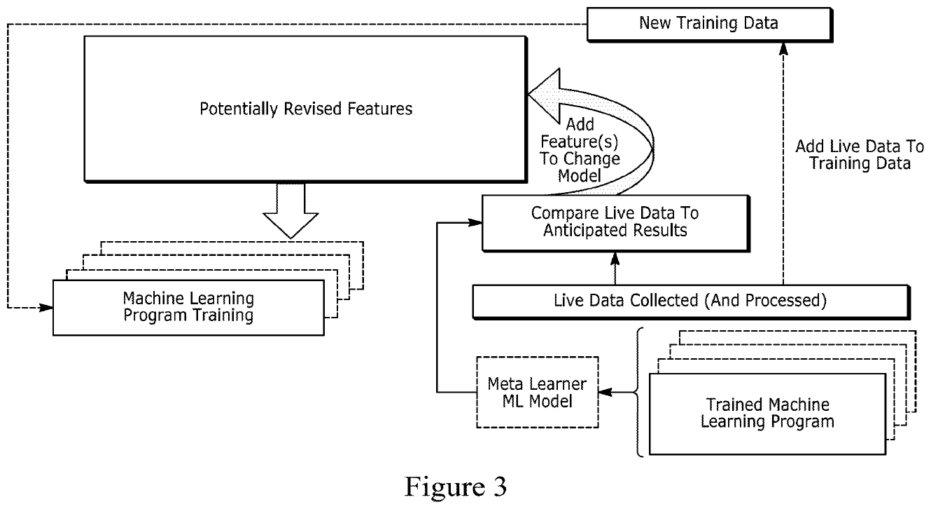

As illustrated in FIGS. 2 and 3, live data may be collected and processed based on the results of actions recommended by the machine learning system being performed. As recommendations progress, additional data, such as ingredient processing data, and consumer sensory data may be collected and may be added to the features of the model.

In particular, one or more machine learning models are trained (102) using selected phenotypic training data collected (101) from the germplasm 105. Once the model is trained, data is input resulting in a list predicted breeding crosses. At its most basic, predictive breeding cross list 115 is based on the probability of the progeny maximizing genetic values, such as protein content.

### Computing Environment to Support Machine Learning

It should also be noted that the machine learning models, data collection, various logic and/or functions disclosed herein may be enabled using any number of combinations of hardware, firmware, and/or as data and/or instructions embodied in various machine-readable or computer-readable media, in terms of their behavioral, register transfer, logic component, and/or other characteristics. Computer-readable media in which such formatted data and/or instructions may be embodied include, but are not limited to, non-volatile storage media in various forms (e.g., optical, magnetic or semiconductor storage media) and carrier waves that may be used to transfer such formatted data and/or instructions through wireless, optical, or wired signaling media or any combination thereof. Examples of transfers of such formatted data and/or instructions by carrier waves include, but are not limited to, transfers (uploads, downloads, e-mail, etc.) over the Internet and/or other computer networks via one or more data transfer protocols (e.g., HTTP, FTP, SMTP, and so on).

Aspects of the methods and systems described herein, such as the logic or machine learning models, may be implemented as functionality programmed into any of a variety of circuitry, including programmable logic devices (“PLDs”), such as field programmable gate arrays (“FPGAs”), programmable array logic (“PAL”) devices, electrically programmable logic and memory devices and standard cell-based devices, as well as application specific integrated circuits. Some other possibilities for implementing aspects include: memory devices, microcontrollers with memory (such as EEPROM), embedded microprocessors, firmware, software, etc. Furthermore, aspects may be embodied in microprocessors having software-based circuit emulation, discrete logic (sequential and combinatorial), custom devices, fuzzy (neural) logic, quantum devices, and hybrids of any of the above device types. The underlying device technologies may be provided in a variety of component types, e.g., metal-oxide semiconductor field-effect transistor (“MOSFET”) technologies like complementary metal-oxide semiconductor (“CMOS”), bipolar technologies like emitter-coupled logic (“ECL”), polymer technologies (e.g., silicon-conjugated polymer and metal-conjugated polymer-metal structures), mixed analog and digital, and so on.

Aspects of the methods and systems disclosed herein may be embodied and/or executed by the logic of the processes described herein, which may also be embodied in the form of software instructions and/or firmware that may be executed on any appropriate hardware. For example, logic embodied in the form of software instructions and/or firmware may be executed on a dedicated system or systems, on a personal computer system, on a distributed processing computer system, and/or the like. In some embodiments, logic may be implemented in a stand-alone environment operating on a single computer system and/or logic may be implemented in a networked environment such as a distributed system using multiple computers and/or processors, for example.

Aspects of the methods and systems described herein may also be implemented on an illustrative system 400, depicted in association with FIG. 4. In particular, system 400 may comprise a user devices 410a-n, server 460, and network 450.

The user device 410 of the system 400 may include various components including, but not limited to, one or more input devices 411, one or more output devices 412, one or more processors 420, a network interface device 425 capable of interfacing with the network 450, one or more non-transitory memories 430 storing processor executable code and/or software application(s), for example including, a web browser capable of accessing a website and/or communicating information and/or data over the network, and/or the like. The memory 430 may also store an application (not shown) that, when executed by the processor 420 causes the user device 410 to provide the functionality of the various systems and methods described the present specification, as would be understood by those of ordinary skill in the art having the present specification, drawings, and claims before them.

The input device 411 may be capable of receiving information input from the user and/or processor 420, and transmitting such information to other components of the user device 410 and/or the network 450. The input device 411 may include, but are not limited to, implementation as a keyboard, touchscreen, mouse, trackball, microphone, remote control, and combinations thereof, for example.

The output device 412 may be capable of outputting information in a form perceivable by the user and/or processor 420. For example, implementations of the output device 412 may include, but are not limited to, a computer monitor, a screen, a touchscreen, an audio speaker, a website, and combinations thereof, for example. It is to be understood that in some exemplary embodiments, the input device 411 and the output device 412 may be implemented as a single device, such as, for example, a computer touchscreen. It is to be further understood that as used herein the term “user” is not limited to a human being, and may comprise, a computer, a server, a website, a processor, a network interface, a user terminal, and combinations thereof, for example.

The server 460 of the system 400 may include various components including, but not limited to, one or more input devices 461, one or more output devices 462, one or more processors 470, a network interface device 475 capable of interfacing with the network 450, and one or more non-transitory memories 480 for storing data structures/tables (including those of database 485) that may be used by the system 400 and particularly server 460 to perform the functions and procedures set forth herein. The memory 480 may also store an application/program store 481 that, when executed by the processor 470 causes the server 460 to provide the functionality of the systems and methods disclosed in the present application.

As shown in FIG. 4, the server 460 may include a single processor or multiple processors working together or independently to execute the program logic 481 stored in the memory 480 as described herein. It is to be understood, that in certain embodiments using more than one processor 470, the processors 470 may be located remotely from one another, located in the same location, or comprising a unitary multi-core processor. The processors 470 may be capable of reading and/or executing processor executable code and/or capable of creating, manipulating, retrieving, altering, and/or storing data structures and data tables (including those of database 485) into the memory 480.

Exemplary embodiments of the processor 470 may be include, but are not limited to, a digital signal processor (DSP), a central processing unit (CPU), a field programmable gate array (FPGA), a microprocessor, a multi-core processor, combinations, thereof, and/or the like, for example. The processor 470 may be capable of communicating with the memory 480 via a path (e.g., data bus). The processor 470 may be capable of communicating with the input device 461 and/or the output device 462.

The input device 461 of the server 460 may be capable of receiving information input from the user and/or processor 470, and transmitting such information to other components of the server 460 and/or the network 450. The input device 461 may include, but are not limited to, implementation as a keyboard, touchscreen, mouse, trackball, microphone, remote control, and/or the like and combinations thereof, for example. The input device 461 may be located in the same physical location as the processor 470, or located remotely and/or partially or completely network-based.

The output device 462 of the server 460 may be capable of outputting information in a form perceivable by the user and/or processor 470. For example, implementations of the output device 462 may include, but are not limited to, a computer monitor, a screen, a touchscreen, an audio speaker, a website, a computer, and/or the like and combinations thereof, for example. The output device 462 may be located with the processor 470, or located remotely and/or partially or completely network-based.

The memory 480 stores applications or program logic 481 as well as data structures (including those of database 485) that may be used by the system 400 and particularly server 460. The memory 480 may be implemented as a conventional non-transitory memory, such as for example, random access memory (RAM), CD-ROM, a hard drive, a solid state drive, a flash drive, a memory card, a DVD-ROM, a disk, an optical drive, combinations thereof, and/or the like, for example. In some embodiments, the memory 480 may be located in the same physical location as the server 460, and/or one or more memory 480 may be located remotely from the server 460. For example, the memory 480 may be located remotely from the server 460 and communicate with the processor 470 via the network 450. Additionally, when more than one memory 480 is used, a first memory 480a may be located in the same physical location as the processor 470, and additional memory 480n may be located in a location physically remote from the processor 470. Additionally, the memory 480 may be implemented as a “cloud” non-transitory computer readable storage memory (i.e., one or more memory 480 may be partially or completely based on or accessed using the network 450).

Each element of the server 460 may be partially or completely network-based or cloud-based, and may or may not be located in a single physical location. As used herein, the terms “network-based,” “cloud-based,” and any variations thereof, are intended to include the provision of configurable computational resources on demand via interfacing with a computer and/or computer network, with software and/or data at least partially located on a computer and/or computer network. In other words, the server 460 may or may not be located in single physical location. Additionally, multiple servers 460 may or may not necessarily be located in a single physical location.

Database 485 may comprise one or more data structures and/or data tables stored on non-transitory computer readable storage memory 480 accessible by the processor 470 of the server 460. The database 485 can be a relational database or a non-relational database. Examples of such databases include, but are not limited to: DB2®, Microsoft® Access, Microsoft® SQL Server, Oracle®, mySQL, PostgreSQL, MongoDB, Apache Cassandra, and the like. It should be understood that these examples have been provided for the purposes of illustration only and should not be construed as limiting the presently disclosed inventive concepts. The database 485 can be centralized or distributed across multiple systems.

While particular embodiments of the present invention have been shown and described, it should be noted that changes and modifications may be made without departing from the presently disclosed inventive concepts in its broader aspects and, therefore, the aim in the appended claims is to cover all such changes and modifications as fall within the true spirit and scope of this invention.

What is claimed is:

1. A method for training a machine-learning model for predictive plant breeding using phenomic selection based on diverse data streams to predict grain composition comprising:
collecting, with a processor, training data, stored in a database, from the group consisting essentially of phenomic data;
selecting, with the processor, a machine learning model based on the training data, the machine learning model selected from the group comprising supervised learning models, unsupervised learning models, and combinations thereof;
training, with the processor, the machine learning model using the training data from the database; and
inputting, via the processor, a new set of phenotypic data from a plurality of grain bearing plants into the trained machine learning model to generate a predictive breeding crosses list ranked on an aggregate probability that a progeny of the cross will exhibit one or more desired phenotypic characteristics.

2. The method according to claim 1 wherein the phenomic data is selected from the group comprising: seed count, seed size, seed weight, and NIR spectra reflectance data from seed/grain.

3. The method according to claim 2 wherein the phenomic data further comprises analytical measurements of seed composition.

4. The method according to claim 3 wherein the phenomic data is further selected from the group comprising: plant height, plant architecture, pod count, leaf size, photosynthetic capacity, root density, and days at each developmental stage.

5. The method according to claim 4 wherein the collecting of training data further comprises gathering spectral reflectance imaging from overall plants, the phenomic data is further selected from the group comprising NDVI, NDRE, and senescence rate.

6. The method according to claim 1 wherein the phenomic data comprises analytical measurements of seed composition.

7. The method according to claim 1 wherein the phenomic data is selected from the group comprising: plant height, plant architecture, pod count, leaf size, photosynthetic capacity, root density, and days at each developmental stage.

8. The method according to claim 1 wherein the collecting of training data further comprises gathering spectral reflectance imaging from overall plants, the phenomic data is selected from the group comprising NDVI, NDRE, and senescence rate.

9. The method according to claim 1 wherein the machine learning model comprises a plurality of stacked ML models.

10. The method according to claim 9 further comprising mediating between the plurality of stacked ML models to produce the aggregated predictive breeding crosses list.
